(19) United States
(12) Patent Application Publication
(10) Pub. No.: US 2025/0387047 A1
(43) Pub. Date: Dec. 25, 2025

(54) SYSTEM AND METHOD FOR PROBABILISTIC ESTIMATION OF CADENCE AND WALKING SPEED

(71) Applicant: University of South Carolina, Columbia, SC (US)

(72) Inventors: Juan Caicedo, Columbia, SC (US); Yohanna Mejia Cruz, Columbia, SC (US); Zhaoshuo Jiang, San Francisco, CA (US)

(73) Assignee: University of South Carolina, Columbia, SC (US)

(21) Appl. No.: 19/244,651

(22) Filed: Jun. 20, 2025

Related U.S. Application Data
(60) Provisional application No. 63/662,868, filed on Jun. 21, 2024.

(51) Int. Cl. A61B 5/11; G08B 21/04

(52) U.S. Cl. CPC A61B 5/112; G08B 21/0423

(57) ABSTRACT

The system uses a probabilistic approach to extract cadence and walking speed from sensor data. This approach prevents the possibility of missing step events in the acceleration signal due to direct source collection or signal enhancement.

### CROSS-REFERENCE TO RELATED APPLICATIONS

This application claims the benefit of prior-filed, co-pending U.S. Provisional Patent Application No. 63/662,868, filed on Jun. 21, 2024, the contents of which are incorporated herein by reference in their entirety.

### STATEMENT OF GOVERNMENT INTEREST

This invention was made with government support under R01 AG067395 awarded by the National Institutes of Health. The government has certain rights in the invention.

### BACKGROUND OF THE INVENTION

Walking patterns provide valuable insights into human health. Gait parameters, including walking speed and cadence, are robust predictors of survival, all-cause mortality, fall risk, physical activity, physical functional decline, and post-acute care setting. Furthermore, deviations from baseline gait patterns can indicate aging, walking capabilities, cognition, and other health-related markers.

Previous studies have correlated gait parameters with changes in health status. For example, a study involving 5,000 adults with a median age of 70.6 years showed that a cadence exceeding 100 steps/minute could be associated with a fifteen-year increase in survival, while a cadence below 100 steps/minute could indicate a ten-year increase. Additionally, a cadence exceeding 100 steps/minute predicted a 21% reduction in all-cause mortality, with each additional ten steps further reducing mortality by 4%.

Walking speed, on the other hand, is a reliable clinical marker across various disease populations. Measurements taken over a 4-meter distance reflect changes in health conditions beyond measurement errors, with variations of approximately 0.11 m/s for medium-speed walkers and 0.14 m/s for fast-speed walkers. Research also shows that increases in walking speed are proportional to changes in cadence, step length, and metabolic intensity.

Interpreting the variability of reported gait measurements and their interplay presents significant challenges in correlating them with changes in health status. This variability includes systematic errors, different technologies used for assessment, random errors during assessment, biased estimations, and inherent variations in subjects' patterns. Typically, gait parameters are measured by having individuals walk on the floor in a straight line or on a treadmill for a specific time; which, as controlled measurements, may not accurately reflect natural gait variations. The Hawthorne effect, the subjective influence of perceived observation, can introduce biases and increase measurement variability, affecting the identification of correlations between gait parameters and health changes.

Unlike controlled clinical settings, home environments reflect individuals' daily challenges, such as varying terrain and environmental conditions, which can impact gait patterns. Conducting assessments in familiar settings allows for a more comprehensive analysis of gait variability and its relation to health changes. Performing assessments at home holds promise for reducing the burden on individuals and healthcare systems, enabling more frequent monitoring. This can be particularly beneficial for longitudinal studies or tracking disease progression, such as Parkinson's or Alzheimer's disease. However, at-home assessments using the most popular available technologies face privacy concerns and challenges related to compliance with device usage, particularly in the case of wearable technologies and patients with cognitive disorders. Forgetfulness or inconsistent use of these devices can impact the ability to provide an accurate snapshot of individuals' gait changes. The measurement of floor vibrations is one of the newest methodologies to identify individuals' walking patterns from the structure's response. Floor vibrations caused by the impact of footsteps during walking create deformations in the floor that sensors like accelerometers or geophones can detect. Once step events are correctly identified from the measurements (e.g., acceleration signals), valuable information can be extracted from complete walking cycles. Various techniques, including the time of arrival methods (ToA), force estimation methods, signal-energy-based algorithms, and transfer learning, have been developed to address challenges associated with event extraction, such as wave dispersion or low signal-to-noise ratios (SNR), which refers to the signal of interest being buried by noise.

Some studies have explored gait parameter extraction using floor vibrations as a sort of stopwatch, measuring the time between the first and last events identified in a controlled setting where the walking distance is known. Others have addressed gait balance symmetry using ground reaction forces with ToA methods employed for localization. However, these methods rely on available energy dissipation throughout the system, which is sensitive to multipath effects. These effects, where signals reflect and arrive via multiple paths, can cause inaccuracies propagating through the estimations. Additionally, the energy dissipation throughout the system requires step events to be reachable by the receiver at all times. This can be problematic if the energy is too low, leading to the complete removal of the event from the signal during filtering. Existing energy-based vibro-localization methods often do not consider the uncertainty of the localization success or failure by providing a measure of the reliability of the collected data, which is imperative in uncontrolled scenarios, especially if there is missing information. ToA methods also require sensor synchronization, which can be challenging in real-home settings where the area to cover is more significant than a hallway or if the furniture is completely rearranged within the home. Studies that address obstructions, such as, may heavily depend on sensor placement and the structural characteristics of the building, impacting the reliability of the localization results. These methods may also require sophisticated signal processing algorithms and computational resources, making real-time deployment challenging in resource-constrained environments.

Some of the most advanced techniques for step localization, accounting for localization uncertainty, are presented in. Although these techniques have yet to be tested in unattended scenarios, they offer a probabilistic approach to localization that can enhance gait extraction. As previous research has shown, floor vibrations present a unique opportunity to advance at-home gait assessments compared to other technologies, owing to their non-intrusive and unobtrusive monitoring nature. However, implementation remains in its early stages and faces significant challenges, particularly in event identification within unattended environments. For instance, the acceleration signal presented in FIG. 1 contains gait data, documenting a record of walking-induced vibrations. From the acceleration data collected, incomplete step events identification, represented by the blue markers, and correct step events identification, represented by the dashed line, provide two completely different estimations of cadence, namely 105 steps/min and 120 steps/min, with a difference of 13%. Such discrepancies can pose problems when attempting to correlate changes in health status with changes in cadence. Tracking all events from an acceleration record containing gait information is not always possible, especially if the person walks away from a sensor; thus requiring an estimation of cadence that can account for the missing information.

Similarly, methodologies aiming to remove noise contributions may inadvertently eliminate the event altogether, especially when the event's frequency band is similar to that of the noise.

There is an unmet need in the art for a method utilizing a probabilistic approach to extract cadence and walking speed to prevent the possibility of missing step events in the acceleration signal due to direct source collection or signal enhancement.

### BRIEF SUMMARY OF THE INVENTION

A method for probabilistic estimation of cadence and walking speed comprises the steps of gathering at least one sensor data set from at least one complete walking cycle using a sensor system, calculating at least one value for cadence from the at least one sensor data set, calculating at least one value for walking speed, calculating uncertainty of the at least one value for cadence, and calculating uncertainty of the at least one value for walking speed.

A system for probabilistic estimation of cadence and walking speed comprises a memory comprising computer readable instructions and a processor configured to read the computer readable instructions that when executed causes the system to perform the above method.

A non-transitory computer readable medium comprises computer readable code to perform probabilistic estimation of cadence and walking speed that when executed by a processor, causes the processor to perform the above method.

The objects and advantages will appear more fully from the following detailed description made in conjunction with the accompanying drawings.

### BRIEF DESCRIPTION OF THE SEVERAL VIEWS OF THE DRAWINGS

FIG. 1 illustrates an embodiment of the system for probabilistic estimation of cadence and walking speed.

FIG. 2 illustrates a flowchart of the method for probabilistic estimation of cadence and walking speed.

FIG. 3 depicts an example diagram of a computer system for probabilistic estimation of cadence and walking speed.

It should be understood that, for clarity, not all elements are necessarily labeled in all drawings. Lack of labeling in a figure should not be interpreted as lack of a feature.

### DETAILED DESCRIPTION OF THE INVENTION

In the present description, certain terms have been used for brevity, clearness and understanding. No unnecessary limitations are to be applied therefrom beyond the requirement of the prior art because such terms are used for descriptive purposes only and are intended to be broadly construed. The different devices, systems, and/or methods described herein may be used alone or in combination with other devices, systems, and/or methods. Dimensions and materials identified in the drawings and applications are by way of example only and are not intended to limit the scope of the claimed invention. Any other dimensions and materials not consistent with the purpose of the present application can also be used. Various equivalents, alternatives and modifications are possible within the scope of the appended claims. Each limitation in the appended claims is intended to invoke interpretation under 35 U.S.C. § 112, sixth paragraph, only if the terms “means for” or “step for” are explicitly recited in the respective limitation.

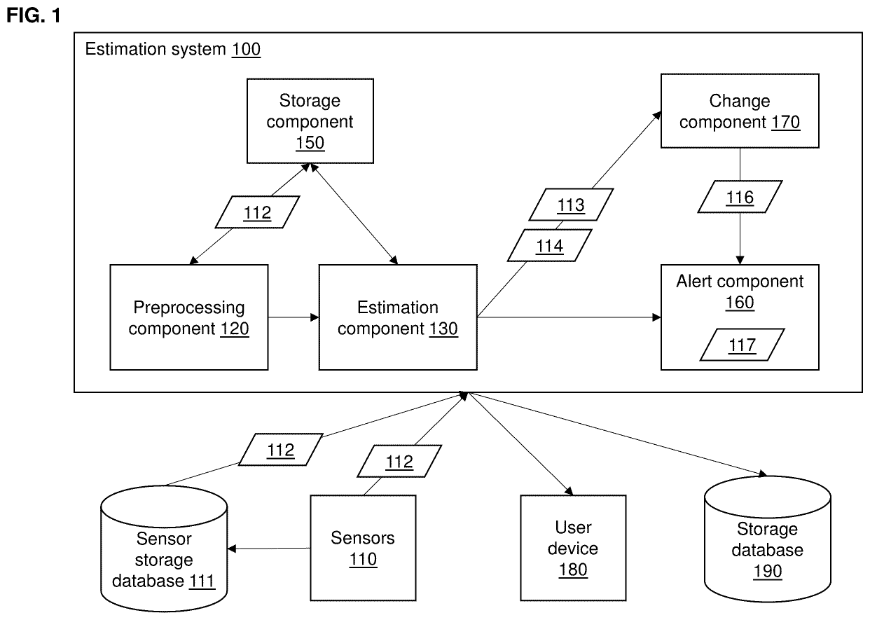

Referring to FIG. 1, in an embodiment, the estimation system 100 may be part of a larger monitoring system (not shown) or may be a separate component integrated with the monitoring system or any other system that stores sensor data, and event data and/or metrics pertaining to sensor data. The system 100 interacts with a sensor system 110 and/or a sensor storage database 111 to receive sensor data sets 112 for estimating cadence (Ω) 113 and walking speed (Π) 114 for walking cycles which are detected by the sensors throughout the sensor system 110. The system 100 includes a preprocessing component 120 to prepare sensor data sets 112, an estimation component 130 to determine the cadence (Ω) 113 and walking speed (Π) 114 based on sensor data sets 112, and estimate the uncertainty of the cadence 113 and walking speed 114, an optional storage component 150, an optional alert component 160 for triggering an alert if a threshold is missed or exceeded, and an optional change component 170 for calculating changes in cadence 113 and/or walking speed 114, also known as “ruptures.” Each of these components will be described in greater detail below. Employees or other members of the entity utilizing the system (hereinafter users) may interact with the system 100. The system 100 optionally includes one or more user devices 180 useable by users for interacting with the system 100. In an embodiment, the system 100 may be a processor(s) or a combination of a processing system and a storage system with a software component and optional storage database 190.

The sensor system 110 may be part of the monitoring system (not shown) or may be a separate component integrated with the monitoring system or any other system that detects gait parameters. The sensor system 110 comprises at least one sensor about an area, such as, but not limited to, a home or care facility. The sensor is capable of detecting and registering at least three step events around the sensor. In certain embodiments, the sensor may detect spatial data such as, but not limited to, step localization. In various embodiments, the sensor may detect vibration, sound, or some combination thereof. In various embodiments, the sensor may be an accelerometer, a geophone, a microphone sensor, or some combination thereof.

A step event is generated by a person causing changes to the information or data gathered by the sensor (e.g., an accelerometer registers acceleration, a geophone detects vibration, a microphone detects sound of a footfall, etc.). In various embodiments, the sensor system 110 records and stores sensor data sets 112 related to all step events generated by the sensors in a sensor storage database 111. The sensor system 110 is also capable of transmitting sensor data sets 112 generated by the sensor in real-time to the system 100.

In an embodiment, each sensor data set 112 may be represented as a data structure including fields for all associated information and data generated by the sensor associated with the step event. Such data structures for each sensor data set 112 comprises a data field for data indicating an instrumentality that caused the related sensor to indicate a change based on sensor function (e.g., acceleration for an accelerometer). In an embodiment, each sensor data set 112 may be represented as an object, including attributes for all associated information and data generated by the sensor associated with the step event. It should be understood that these are merely examples and that any appropriate structure for associating the data collected for the sensor data sets 112 may be used.

The system 100 includes a preprocessing component 120 to receive and transform the sensor data sets 112. The preprocessing component 120 may receive sensor data sets 112 from the sensor event storage database 111 in batches or may receive sensor data sets 112 in real-time or near real-time directly from the sensor system 110 when the step event is generated. In an embodiment, the sensor data sets 112 received by the preprocessing component 120 are all step events collected by all sensors for a given period of time, such as, but not limited an hour, a day, or a month. In an embodiment, the sensor data sets 112 received by the preprocessing component 120 are all events generated in a given time period and/or for a subset of given sensors and/or regions throughout the sensor system 110. By way of non-limiting example, sensor data sets 112 from the sensor or sensors 110 covering a portion of a room, a single room, or a set of rooms may be received. By way of non-limiting example, sensor data sets 112 may be received for a time period within a single day, or for a single day, a week, a month, or any other time period.

The preprocessing component 120 performs processing procedures on the sensor data sets 112. In an embodiment, the procedure is application of filtering methodologies to distinguish the gait signal from the ambient noise. In an embodiment, the preprocessing component 120 may be a processor or a combination of a processing system and a storage system with a software component and optional storage.

The system 100 includes an estimation component 130 for calculating at least one value for cadence 113 and/or walking speed 114 from the at least one sensor data set 112 as disclosed below. In certain embodiments, the estimation component 130 also receives informative priors for the α and β parameters of the walking speed for each sensor data set 112. The informative priors are set at the beginning of the process by a user. α and β are unknown random variables representing slope and intercept, respectively, of the proportional relationship between cadence and walking speed.

The system 100 also uses the estimation component 130 for calculating at least one value for accuracy of the calculated cadence 113 and/or walking speed 114. The estimation component 130 uses Gaussian likelihood to calculate the probable uncertainty 115 of the calculations for cadence 113 and/or walking speed 114 as disclosed below.

The system 100 optionally includes a change component 170 for calculating at least one change value 116 for the cadence 113 and/or walking speed 114. over time. In one embodiment, the change value 116 is a simple differential magnitude. In other embodiments, the change value 116 may be an average change over time, or may be weighted to provide greater or lesser importance to more recent values for cadence 113 and/or walking speed 114.

The system 100 optionally includes an alert component 160 for comparing at least one of the cadence 113, walking speed 114, probable uncertainty 115, and/or change value 116 to at least one threshold value 117. Depending upon the given compared value, an alert may be transmitted by the alert component 160 if the compared value exceeds or fails to exceed the threshold value 117.

Optionally, the values for the cadence 113, walking speed 114, probable uncertainty 115, change value 116, and/or threshold value 117 may be stored in a storage component 150 for later use. The system 100 may also include a user device 180 for displaying sensor the cadence 113, walking speed 114, probable uncertainty 115, change value 116, and/or threshold value 117, and for allowing users to interact with the system 100. The system 100 may further include a storage database 190 for storing the cadence 113, walking speed 114, probable uncertainty 115, change value 116, threshold value 117, and any other values utilized or generated by the system 100.

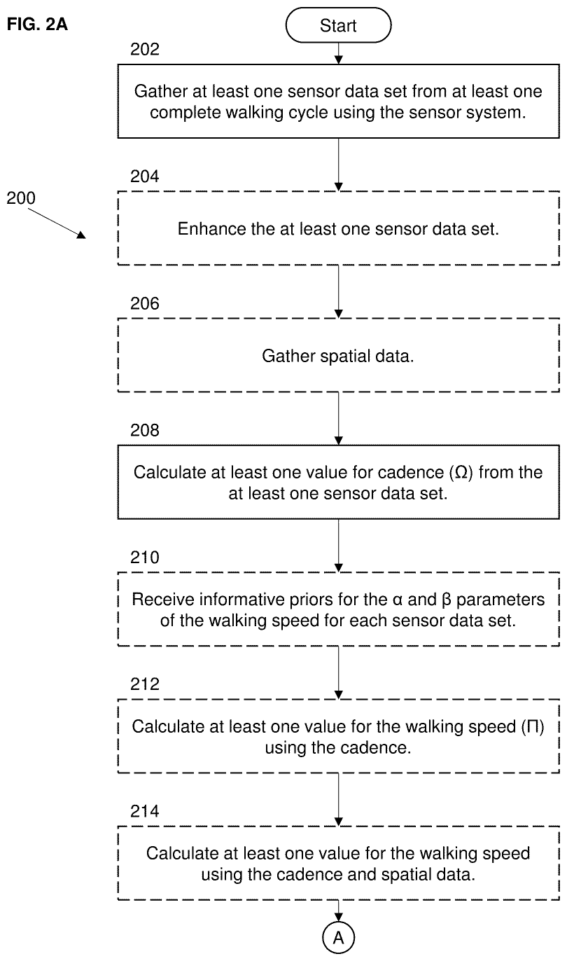

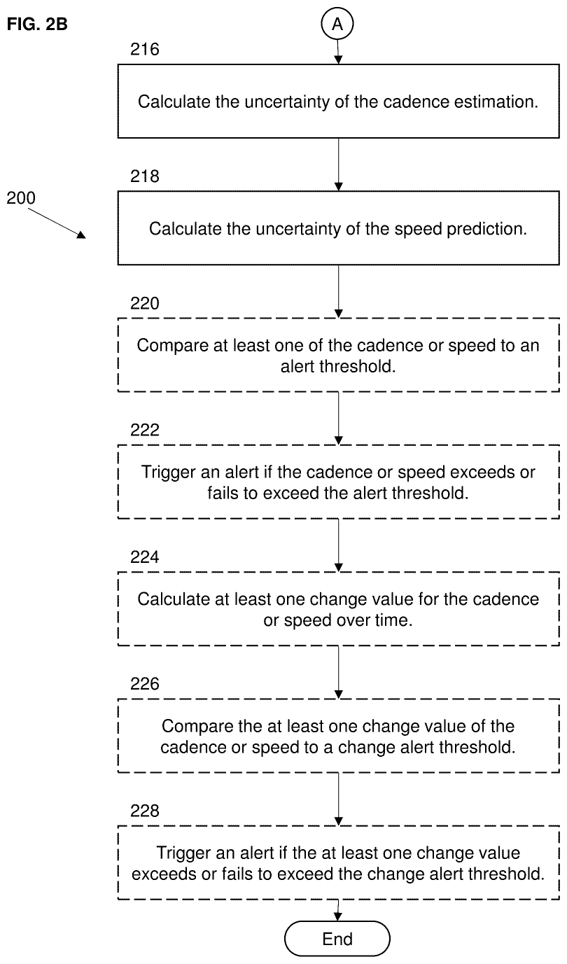

FIG. 2 illustrates a flowchart of the method 200 for probabilistic estimation of cadence and walking speed. It is to be understood that the numbering and sequencing of the blocks are for reference only; blocks or sequences of blocks may be performed out of order or repeated.

In block 202, the method gathers at least one sensor data set comprising gait parameters from at least one complete walking cycle using the sensor system. The complete walking cycle includes at least three step events detected by the sensor system.

In optional block 204, the method enhances the at least one sensor data set using the preprocessing component. Such enhancement may include application of filtering methodologies to distinguish the step events from ambient noise.

In optional block 206, the method gathers spatial data. In one embodiment, the spatial data set is step localization, allowing a more accurate estimation of an individual's walking speed by directly accounting for step length, which is influenced by subject-specific characteristics such as age or height.

In block 208, the method calculates at least one value for cadence (Ω) from the at least one sensor data set using the equation:

$$Ω = \frac{60}{{t}_{{s}_{i}} - {t}_{{s}_{0}}}$$

where tₛᵢ represents the time of the i-th step from i={1, 2, . . . n} of n steps and tₛ₀ the time of the first step.

In optional block 210, the method receives informative priors for the α and β parameters of the walking speed for each sensor data set. α and β are unknown random variables representing slope and intercept, respectively, of the proportional relationship between cadence and walking speed.

In optional block 212, the method calculates at least one value for the walking speed (I) using the cadence in the equation:

$$\prod = αΩ - β$$

In optional block 214, the method calculates at least one value for the walking speed using the cadence and spatial data in the equation:

$$\prod = S Ω$$

where S is step length.

In block 216, the method calculates the uncertainty of the cadence estimation. In one embodiment, the calculation uses the Gaussian likelihood equation:

$$P ( {D}_{1} \vert θ M ) = {\prod }_{i = 1}^{n} \frac{1}{{σ}_{t} \sqrt{2 π}} \times exp [ - \frac{1}{2} \frac{{( {t}_{i} - {t}_{si} ( θ M ) )}^{2}}{{σ}_{t}^{2}} ]$$

where σₜ is a random variable defined by exponential prior distributions as σₜ˜f(x|λ=1/Δt_{d}), where Δt_{d} represents the lowest time interval between detected steps events from the acceleration signal.

In block 218, the method calculates the uncertainty of the speed prediction. In one embodiment, the calculation uses the Gaussian likelihood equation:

$$P ( {D}_{2} \vert θ M ) = {\prod }_{i = 1}^{n} \frac{1}{{σ}_{ws} \sqrt{2 π}} \times exp [ - \frac{1}{2} \frac{{( {\hat{\prod }}_{i} - {\overline{\prod }}_{i} ( θ M ) )}^{2}}{{σ}_{ws}^{2}} ]$$

where σ_{ws} is a random variable defined by exponential prior distributions as σ_{ws}˜f(x|λ=10).

In optional block 220, the method compares at least one of the cadence or speed to an alert threshold.

In optional block 222, the method triggers an alert if the cadence or speed exceeds or fails to exceed the alert threshold.

In optional block 224, the method calculates at least one change value for the cadence or speed over time.

In optional block 226, the method compares the at least one change value of the cadence or speed to a change alert threshold.

In optional block 228, the method triggers an alert if the at least one change value of the cadence or speed exceeds or fails to exceed the change alert threshold.

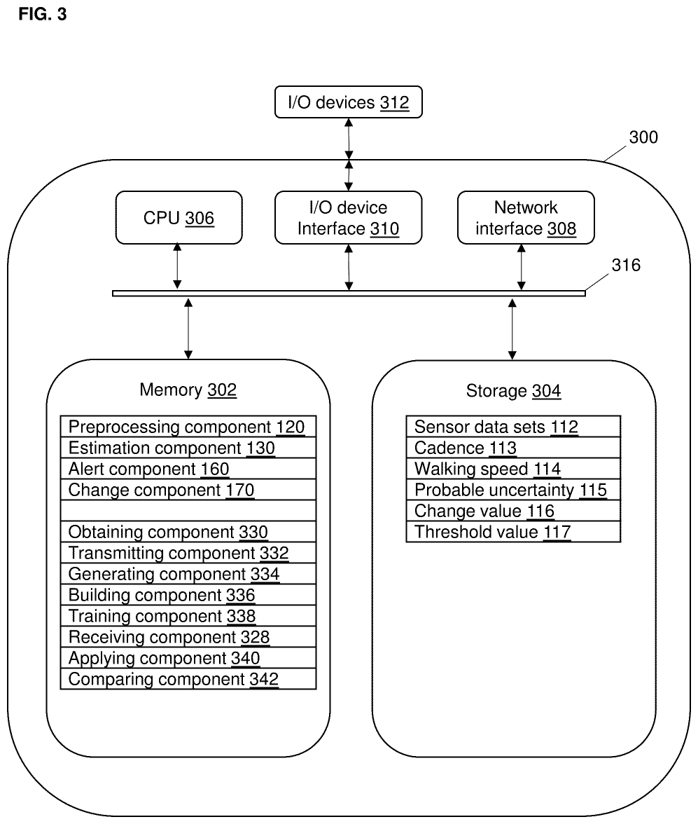

FIG. 3 depicts an example diagram of a computer system 300 for probabilistic estimation of cadence and walking speed that may include the kinds of software programs, data stores, hardware, and interfaces that can implement an estimation system 100 as disclosed herein and according to certain embodiments. The computing system 300 may be used to implement embodiments of portions of the estimation system 100 and/or in carrying out embodiments of method 200. The computing system 300 may be part of or connected to an overarching customer service center system.

As shown, the computer system 300 includes, without limitation, a memory 302, a storage 304, a central processing unit (CPU) 306, and a network interface 308, each connected to a bus 316. The computing system 300 may also include an input/output (I/O) device interface 310 connecting I/O devices 312 (e.g., keyboard, display, and mouse devices) and/or a network interface 308 to the computing system 300. Further, the computing elements shown in computer system 300 may correspond to a physical computing system (e.g., a system in a data center), a virtual computing instance executing within a computing cloud, and/or several physical computing systems located in several physical locations connected through any combination of networks and/or computing clouds.

Computing system 300 is a specialized system specifically designed to perform the steps and actions necessary to execute method 200 and estimation system 100. While some of the component options for computing system 300 may include components prevalent in other computing systems, computing system 300 is a specialized computing system specifically capable of performing the steps and processes described herein.

The CPU 306 retrieves, loads, and executes programming instructions stored in memory 302. The bus 316 is used to transmit programming instructions and application data between the CPU 306, I/O interface 310, network interface 308, and memory 302. Note, the CPU 306 can comprise a microprocessor and other circuitry that retrieves and executes programming instructions from memory 302. CPU 306 can be implemented within a single processing element (which may include multiple processing cores) but can also be distributed across multiple processing elements (with or without multiple processing cores) or sub-systems that cooperate in existing program instructions. Examples of CPUs 306 include central processing units, application-specific processors, and logic devices, as well as any other type of processing device, a combination of processing devices, or variations thereof. While there are a number of processing devices available to compromise the CPU 306, the processing devices used for the CPU 306 are particular to this system and are specifically capable of performing the processing necessary to execute method 200 and estimation system 100.

The memory 302 can comprise any memory media readable by CPU 306 that is capable of storing programming instructions and able to meet the needs of the computing system 300 and execute the programming instructions required for method 200 and estimation system 100. Memory 302 is generally included to be representative of a random-access memory. In addition, memory 302 may include volatile and non-volatile, removable, and non-removable media implemented in any method or technology for storage of information, such as computer-readable instructions or program components. The memory 302 may be implemented as a single memory device but may also be implemented across multiple memory devices or sub-systems. The memory 302 can further include additional elements, such as a controller capable of communicating with the CPU 306.

Illustratively, the memory includes multiple sets of programming instructions for performing the functions of the estimation system 100 and method 200, including, but not limited to, preprocessing component 120, estimation component 130, storage component 150, alert component 160, and change component 170, all of which are discussed in greater detail herein. Illustratively, the memory may also include an obtaining component 330, a transmitting component 332, a generating component 334, a building component 336, a training component 338, a receiving component 328, an applying component 340, and a comparing component 342. Although memory 302, as depicted in FIG. 3, includes twelve sets of programming instruction components in the present example, it should be understood that one or more components could perform single- or multi-component functions. It is also contemplated that these components of computing system 300 may be operating in a number of physical locations.

The storage 304 can comprise any storage media readable by CPU 306 and is capable of storing data that is able to meet the needs of computing system 300 and store the data required for method 200 and estimation system 100. The storage 304 may be a disk drive or flash storage device. The storage 304 may include volatile and non-volatile, removable, and non-removable media implemented in any method or technology for storage of information. Although shown as a single unit, the storage 304 may be a combination of fixed and/or removable storage devices, such as fixed disc drives, removable memory cards, optical storage, network-attached storage (NAS), or a storage area-network (SAN). The storage 304 can further include additional elements, such as a controller capable of communicating with the CPU 306.

Illustratively, the storage 304 may store data such as but not limited to cadence 113, walking speed 114, probable uncertainty 115, change value 116, threshold value 117, and any other values utilized or generated by the system 100, all of which are also discussed in greater detail herein.

Examples of memory and storage media include random access memory, read-only memory, magnetic discs, optical discs, flash memory, virtual memory, and non-virtual memory, magnetic sets, magnetic tape, magnetic disc storage, or other magnetic storage devices, or any other medium which can be used to store the desired software components or information that may be accessed by an instruction execution system, as well as any combination or variation thereof, or any other type of storage medium. In some implementations, one or both of the memory and storage media can be a non-transitory memory and storage media. In some implementations, at least a portion of the memory and storage media may be transitory. Memory and storage media may be incorporated into computing system 300. While many types of memory and storage media may be incorporated into computing system 300, the memory and storage media used is capable of executing the storage requirements of method 200 and estimation system 100 as described herein.

The I/O interface 310 allows computing system 300 to interface with I/O devices 312. I/O devices 312 can include one or more user devices 180, sensor systems 110, graphical user interfaces, desktops, a mouse, a keyboard, a voice input device, a touch input device for receiving a gesture from a user, a motion input device for detecting non-touch gestures and other motions by a user, and other comparable I/O devices and associated processing elements capable of receiving input. The I/O devices 312 through the user devices 160 are also integrated into the system allowing users to access the sensor system 110, the security management system, telephone system, internet system, and a text communications system, among other systems. I/O devices 312 can also include devices such as a video display or graphical display and other comparable I/O devices and associated processing elements capable of providing output. Speakers, printers, haptic devices, or other types of output devices may also be included in the I/O device 312.

A user can communicate with computing system 300 through the I/O device 312 in order to view cadence 113, walking speed 114, probable uncertainty 115, change value 116, threshold value 117, and any other values utilized or generated by the system 100, receive alerts from alert component 160, or complete any number of other tasks the user may want to complete with computing system 300. I/O devices 312 can receive and output data such as but not limited to cadence 113, walking speed 114, probable uncertainty 115, change value 116, threshold value 117, and any other values utilized or generated by the system 100.

As described in further detail herein, computing system 300 may receive and transmit data from and to the network interface 308. In embodiments, the network interface 308 operates to send and/or receive data, such as but not limited to, cadence 113, walking speed 114, probable uncertainty 115, change value 116, threshold value 117, and any other values utilized or generated by the system 100 to/from other devices and/or systems to which computing system 300 is communicatively connected, and to receive and process interactions as described in greater detail above.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although certain implementations may refer to utilizing aspects of the presently disclosed subject matter in the context of one or more stand-alone computer systems, the subject matter is not so limited but rather may be implemented in connection with any computing environment, such as a network or distributed computing environment. Still further, aspects of the presently disclosed subject matter may be implemented in or across a plurality of processing chips or devices, and storage may similarly be affected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

In the foregoing description, certain terms have been used for brevity, clearness, and understanding. No unnecessary limitations are to be inferred therefrom beyond the requirement of the prior art because such terms are used for descriptive purposes and are intended to be broadly construed. The different configurations, systems, and method steps described herein may be used alone or in combination with other configurations, systems, and method steps. It is to be expected that various equivalents, alternatives, and modifications are possible within the scope of the foregoing description.

What is claimed is:

1. A method for probabilistic estimation of cadence and walking speed, comprising:
gathering at least one sensor data set from at least one complete walking cycle using a sensor system;
calculating at least one value for cadence from the at least one sensor data set;
calculating at least one value for walking speed;
calculating uncertainty of the at least one value for cadence; and
calculating uncertainty of the at least one value for walking speed.

2. The method of claim 1, wherein the complete walking cycle includes at least three step events detected by the sensor system.

3. The method of claim 1, further comprising enhancing the at least one sensor data set.

4. The method of claim 3, wherein enhancing the at least one sensor data set comprises application of filtering methodologies to distinguish step events from ambient noise.

5. The method of claim 1, wherein the at least one value for cadence (0) is calculated from the at least one sensor data set using the equation:
$$Ω = \frac{60}{{t}_{{s}_{i}} - {t}_{{s}_{0}}}$$
where tsi represents the time of the i-th step from i={1, 2, . . . n} of n steps and ts0 the time of the first step.

6. The method of claim 1, further comprising receiving informative priors for at least one α parameter and at least one β parameter of the at least one value for walking speed for the at least one sensor data set.

7. The method of claim 6, wherein the α and β parameters are unknown random variables representing slope and intercept, respectively, of a proportional relationship between the at least one value for cadence and the at least one value for walking speed.

8. The method of claim 6, wherein the at least one value for the walking speed (π) is calculated using the at least one value for cadence in the equation:
$$\prod = αω - β$$

9. The method of claim 1, further comprising gathering spatial data.

10. The method of claim 9, wherein the spatial data set is step localization.

11. The method of claim 9, wherein the at least one value for the walking speed is calculated using the at least one value for cadence and spatial data in the equation:
$$\prod = S ω$$
where S is step length.

12. The method of claim 1, wherein calculating uncertainty of the at least one value for cadence is calculated using the Gaussian likelihood equation:
$$P ( {D}_{1} \vert θ M ) = {\prod }_{i = 1}^{n} \frac{1}{{σ}_{t} \sqrt{2 π}} \times Exp [ - \frac{1}{2} \frac{{( {t}_{i} - {t}_{si} ( θ M ) )}^{2}}{{σ}_{t}^{2}} ]$$
where σt is a random variable defined by exponential prior distributions as σt˜f(x|λ=1/δtd), where δtd represents the lowest time interval between detected steps events from the acceleration signal.

13. The method of claim 1, wherein calculating uncertainty of the at least one value for the walking speed is calculated using the Gaussian likelihood equation:
$$P ( {D}_{2} \vert θ M ) = {\prod }_{i = 1}^{n} \frac{1}{{σ}_{ws} \sqrt{2 π}} \times Exp [ - \frac{1}{2} \frac{{( {\hat{\prod }}_{i} - {\overline{\prod }}_{i} ( θ M ) )}^{2}}{{σ}_{ws}^{2}} ]$$
where ows is a random variable defined by exponential prior distributions as σws˜f(x|λ=10).

14. The method of claim 1, further comprising comparing at least one of the of the at least one value for cadence or the at least one value for walking speed to an alert threshold.

15. The method of claim 14, further comprising triggering an alert if the at least one value for cadence or the at least one value for walking speed exceeds or fails to exceed the alert threshold.

16. The method of claim 1, further comprising calculating at least one change value for the at least one value for cadence or the at least one value for walking speed over time.

17. The method of claim 16, further comprising comparing the at least one change value of the at least one value for cadence or the at least one value for walking speed to a change alert threshold.

18. The method of claim 17, further comprising triggering an alert if the at least one change value of the at least one value for cadence or the at least one value for walking speed exceeds or fails to exceed the change alert threshold.

19. A system for probabilistic estimation of cadence and walking speed, comprising:
a memory comprising computer readable instructions;
a processor configured to read the computer readable instructions that when executed causes the system to:
gather at least one sensor data set from at least one complete walking cycle using a sensor system;
calculate at least one value for cadence from the at least one sensor data set;
calculate at least one value for walking speed;
calculate uncertainty of the at least one value for cadence; and
calculate uncertainty of the at least one value for walking speed.

20. A non-transitory computer readable medium comprising:
computer readable code to perform probabilistic estimation of cadence and walking speed that when executed by a processor, causes the processor to:
gather at least one sensor data set from at least one complete walking cycle using a sensor system;
calculate at least one value for cadence from the at least one sensor data set;
calculate at least one value for walking speed;
calculate uncertainty of the at least one value for cadence; and
calculate uncertainty of the at least one value for walking speed.
